(19) United States
(12) Patent Application Publication
(10) Pub. No.: US 2008/0096831 A1
(43) Pub. Date: Apr. 24, 2008

(54) Stabilized azithromycin composition

(76) Inventors: Mohsen Sadatrezaei, Allentown, PA (US); Pablo Davila, East Windsor, NJ (US); Gary Barbera, Medford, NJ (US)

Correspondence Address: SANDOZ INC, 506 CARNEFIE CENTER, PRINCETON, NJ, 08540, US

(21) Appl. No.: 11/881,664

(22) Filed: Jul. 27, 2007

Related U.S. Application Data
(63) Continuation of application No. 10/702,690, filed on Nov. 6, 2003, now abandoned.

(51) Int. Cl. A61K 31/7048; A61P 31/04

(52) U.S. Cl. 514/29

(57) ABSTRACT

A stabilized azithromycin composition comprising azithromycin monohydrate, and about 5 to about 15 weight percent, based on the total weight of the composition, of water. The present inventors have unexpectedly determined that a certain amount of water is necessary to stabilize a pharmaceutical composition comprising azithromycin monohydrate. In addition, the stabilized azithromycin monohydrate compositions do not require an antioxidant.

### FIELD OF THE INVENTION

The present invention provides a stabilized azithromycin composition comprising azithromycin monohydrate, and about 5 weight percent (wt %) to about 15 wt %, based on the total weight of the composition, of water.

### BACKGROUND OF THE INVENTION

Azithromycin monohydrate has the chemical formula [2R-(2R*,3S*,4R*,5R*,8R*, 10R*, 11R*, 12S*, 13S*, 14R*)]-13-[(2,6-dideoxy-3-C-methyl-3-β-methyl-α-L-ribo-hexopyranosyl)oxy]-2-ethyl-3,4,10-trihydroxy-3,5,6,8,10,12,14-heptamethyl-11-[[3,4,6-trideoxy-3-(dimethylamino)-β-D-xylo-hexopyranosyl]oxy]-1-oxa-6-azacyclopentadecan-15-one monohydrate. Azithromycin is a broad spectrum antimicrobial compound derived from erythromycin A. Azithromycin was independently discovered by Kobrehel and Djokic, U.S. Pat. No. 4,517,359; and Bright, U.S. Pat. No. 4,474,768. These patents disclose that azithromycin and certain derivatives thereof possess antimicrobial properties and are accordingly useful as antibiotics.

Azithromycin monohydrate is very hygroscopic and unstable. In particular, the amine group of azithromycin monohydrate is susceptible to oxidation especially when exposed to temperatures above about 25° C. and/or air during manufacturing processes. In addition, pharmaceutical compositions containing azithromycin monohydrate have a tendency to degrade under normal storage conditions. Oxidation and/or degradation of the azithromycin monohydrate may deleteriously effect purity and lead to inaccurate dosage amounts.

U.S. Pat. No. 6,365,574 describes a non-hygroscopic form of azithromycin which is prepared by gradual crystallization of azithromycin from ethanol by the addition of a minimal amount of water to effect crystal formation. The azithromycin ethanolate has an ethanol content of about 1.5-3% and a water content of about 24%.

There continues to be a need for improved azithromycin compositions and methods of manufacturing such compositions in which the tendency for oxidation and/or degradation of the azithromycin is reduced, resulting in more stabilized azithromycin compositions.

### SUMMARY OF THE INVENTION

The invention provides a stabilized azithromycin composition comprising azithromycin monohydrate, and about 5 wt % to about 15 wt %, based on the total weight of the composition, of water. The stabilized azithromycin composition is preferably in the form of a tablet.

According to another aspect, the invention provides a method for preparing a stabilized azithromycin composition comprising mixing azithromycin monohydrate and water to form a stabilized azithromycin composition having a water content from about 5 to about 15 weight percent, based on the total weight of the composition.

According to another aspect, the invention provides a method for preparing a stabilized azithromycin composition comprising mixing azithromycin monohydrate and at least one excipient containing water to form a stabilized azithromycin composition having a water content from about 5 to about 15 weight percent, based on the total weight of the composition.

The present inventors have unexpectedly determined that a certain amount of water is necessary to stabilize a pharmaceutical composition comprising azithromycin monohydrate. In addition, the stabilized azithromycin monohydrate compositions do not require an antioxidant.

### BRIEF DESCRIPTION OF THE DRAWINGS

FIG. 1 is a graph illustrating moisture sorption-desorption isotherm of azithromycin monohydrate granules.

FIG. 2 is a graph illustrating the percent loss on drying (LOD) vs. time for azithromycin monohydrate granules.

FIG. 3 is a graph illustrating the percent LOD vs. time for azithromycin monohydrate granules upon exposure to different humidity levels.

As used herein, “loss on drying” (LOD) refers to the water content of a sample, as determined using methods in U.S. Pharmacopeia Chapter 921.

### DETAILED DESCRIPTION OF THE INVENTION

The invention provides a stabilized azithromycin composition comprising azithromycin monohydrate, and about 5 wt % to about 15 wt %, based on the total weight of the composition, of water. As used herein, “stabilized” means that the formation of impurities is reduced or eliminated. Preferably, the water is present in an amount of from about 5.5 wt % to about 12.4 wt %, more preferably from about 6 wt % to about 8 wt %, based on the total weight of the composition. Most preferably, the water is present in an amount of from about 6 wt % to about 7 wt %.

The azithromycin monohydrate is preferably present in the stabilized azithromycin composition in an amount of from about 0.1 wt % to about 95 wt %, based on the total weight of the composition. More preferably, the azithromycin monohydrate is present in an amount of from about 30 wt % to about 85 wt %, most preferably, from about 50 wt % to about 75 wt %, based on the total weight of the composition.

It is within the scope of the invention to prepare stabilized azithromycin compositions that are “essentially free” of an antioxidant. As used herein, “essentially free” means that the compositions contain less than 5 wt % of an antioxidant, based on the total weight of the composition. Preferably, the compositions contain less than 3 wt %, more preferably less than 1 wt % of an antioxidant.

Optionally, the stabilized azithromycin compositions of the invention may contain an antioxidant. As used herein, “antioxidant” refers to a substance known to inhibit oxidation. Examples of antioxidants include ascorbic acid, sodium ascorbate, calcium ascorbate, ascorbic palmitate, butylated hydroxyanisole, butylated hydroxytoluene, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone and tocopherols, such as vitamin E, and the like, including pharmaceutically acceptable salts and esters of these compounds. If present, the antioxidant is generally used in an amount of from about 0.01 wt % to about 10 wt %, based on the weight of the azithromycin monohydrate.

It is within the scope of the invention for the stabilized azithromycin compositions to include one or more pharmaceutically acceptable excipients. Examples of such excipients are binders, diluents, anti-caking agents, amino acids, fillers, solubilizers, disintegrants, lubricants, emulsifiers, flavorants, solvents, stabilizers, anti-oxidants, anti-adherents, preservatives, electrolytes and glidants. A combination of excipients may also be used. Such excipients are known to those skilled in the art, and thus, only a limited number will be specifically referenced.

Examples of binders include, cellulose derivatives (such as microcrystalline cellulose, methylcellulose, carboxymethylcellulose sodium, hydroxypropyl methylcellulose, hydroxyethyl cellulose, and hydroxypropyl cellulose), polyvidone, polyvinyl pyrrolidone, gelatin, natural gums (such as acacia, tragacanth, guar, and pectin), starch paste, pregelatinized starch, sucrose, corn syrup, polyethylene glycols, and sodium alginate, ammonium calcium alginate, magnesium aluminum silicate, and polyethylene glycols.

Examples of fillers or diluents include, spray-dried or anhydrous lactose, sucrose, dextrose, starch, pregelatinized starch, polyols (such as mannitol, sorbitol, and xylitol), cellulose (such as microcrystalline cellulose), and inorganic salts (such as dibasic calcium phosphate, tribasic calcium phosphate, and calcium sulfate). Preferably the filler is a combination of pregelatinized starch and microcrystalline cellulose.

Examples of disintegrants include, starch and starch derivatives, including cross-linked sodium salt of a carboxymethyl ether of starch (such as sodium starch glycolate), pregelatinized starch (such as Starch 1500), sodium starch glycolate, cross-linked sodium carboxymethyl cellulose (such as Croscarmellose Sodium), cross-linked polyvinylpyrrolidone (such as Crospovidone), and microcrystalline cellulose. A preferred disintegrant is sodium starch glycolate.

Examples of lubricants include vegetable oils (such as corn oil), mineral oils, polyethylene glycols (such as PEG-4000 and PEG-6000), salts of stearic acid (such as calcium stearate, magnesium stearate, and sodium stearyl fumarate), mineral salts (such as talc), inorganic salts (such as sodium chloride), organic salts (such as sodium benzoate, sodium acetate, and sodium oleate), polyvinyl alcohols, sodium lauryl sulfate, and magnesium lauryl sulfate. Preferred lubricants are magnesium stearate, and mixtures of magnesium stearate with sodium lauryl sulfate.

The stabilized azithromycin compositions of the invention are preferably in an oral dosage form, such as but not limited to, tablets, granules, dragees, hard or soft capsules, powders, and multiparticules. Preferably, the dosage form is a tablet. The term “tablet” includes compressed tablets, coated tablets, matrix tablets, osmotic tablets and other forms known in the art.

The stabilized azithromycin compositions may be coated to provide ease of swallowing and an elegant appearance. Examples of polymeric film-coating materials include the following: hydroxypropylmethyl cellulose, hydroxypropyl cellulose, cross-linked polyvinyl pyrrolidone; non-cross linked polyvinylpyrrolidone; hydroxypropylmethyl cellulose phthalate, hydroxypropylmethyl cellulose acetate succinate, cellulose acetate succinate; cellulose acetate phthalate, hydroxypropylmethyl cellulose acetate succinate, cellulose acetate trimellitate, hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene copolymer; polyvinylalcohols; polyoxyethyleneglycols; polyethylene glycol; sodium alginate; galactomannone; carboxypolymethylene; sodium carboxymethyl starch; copolymers of acrylic acid and/or methacrylic acid with a monomer selected from the following: methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate, or octadecyl acrylate, e.g. EUDRAGIT®-L and -S series, such as L100-55, L30D55, L100, S100, L12,5, and S12,5, available from Rohm; polyvinyl acetate; fats; oils; waxes; fatty alcohols; shellac; gluten; ethylacrylate-maleic acid anhydride copolymer; maleic acid anhydride-vinyl methyl ether copolymer; styrol-maleic acid copolymer; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); and polyurethane. A combination of coatings may also be used. A preferred coating is Opadry® which is available from Colorcon Corp.

Conventional tableting processes or methods are employed, e.g., by forming a tablet from a desired blend or mixture of ingredients into the appropriate shape using a conventional tablet press. Tablet formulation and conventional processing techniques have been widely-described.

During the preparation of the stabilized azithromycin compositions, the present inventors have determined that humidity may deleteriously effect the water content of the compositions. For example, the present inventors have determined that in order to maintain a water content or LOD of between 6% and 7% in azithromycin monohydrate granules, a humidity range of between 40% relative humidity (RH) and 70% RH should be maintained during manufacturing operations. Manufacturing operations wherein the composition may be exposed to ambient humidity includes, but is not limited to, transfer of dried granulation from the fluid bed dryer to drums, milling of the dried granulation, final mixing with sodium starch glycolate and magnesium stearate, discharge of the blender into open drums, exposure during tabletting operations, and equilibration of the azithromycin compositions in an open environment.

Preferably the stabilized azithromycin compositions are prepared within a humidity range of 20-99% RH, e.g., 25-90% RH. More preferably, the stabilized azithromycin compositions are prepared within a humidity range of 30-80% RH, most preferably 45-70% RH.

In one embodiment of the invention, a stabilized azithromycin composition is prepared by a method comprising mixing azithromycin monohydrate and water to form a stabilized azithromycin composition having a water content from about 5 to about 15 weight percent, based on the total weight of the composition.

In another embodiment of the invention, a stabilized azithromycin composition is prepared by a method comprising mixing azithromycin monohydrate and at least one excipient containing water to form a stabilized azithromycin composition having a water content from about 5 to about 15 weight percent, based on the total weight of the composition. Examples of excipients which may contain water include, but are not limited to, starch and microcrystalline cellulose.

In another embodiment of the invention, a stabilized azithromycin composition is prepared by a method comprising:
(a) mixing azithromycin monohydrate, and optionally one or more excipients, to form a premix;
(b) adding water, and optionally one or more excipients, to the premix formed in Step (a) to form a mixture;
(c) drying the mixture formed in Step (b), and optionally milling and screening the mixture; and
(d) adding water to the mixture formed in Step (c) to form a stabilized azithromycin composition having a water content from about 5 to about 15 weight percent, based on the total weight of the composition.

Drying techniques include spray-drying, fluid bed drying, flash drying, ring drying, micron drying, tray drying, vacuum drying, radio-frequency drying and microwave drying. A preferred drying technique is fluid bed.

Types of mills which may be used in the invention include, but are not limited to, fluid energy mill, ball mill or rod mill, hammer mill, cutting mill and oscillating granulator. More specifically, suitable mills include, Quadro, Fryma, Glatt Quick Sieve, Fluidaire, Fitzpatrick (Fitz mill), BTS mill and Tomado. A preferred mill is a Fitz mill.

In a further aspect, this invention provides a method for treating a microbial infection, comprising administering to a mammal in need of such treatment, including a human patient, a therapeutically effective amount of the stabilized azithromycin composition in an immediate-release, extended-release or controlled-release oral dosage form.

The following non-limiting examples illustrate further aspects of the invention.

### EXAMPLE 1

### Preparation of a Stabilized Azithromycin Composition

| Ingredient | Amount | |
|---|---|---|
| Azithromycin Monohydrate | 550.0 | mg |
| Pregelatinized Starch NF | 213.0 | mg |
| Microcrystalline Cellulose NF | 57.0 | mg |
| Sodium Lauryl Sulfate NF | 3.0 | mg |
| Colloidal Silicon Dioxide NF | 10.0 | mg |
| Purified Water | q.s. | |
| Sodium Starch Glycolate NF | 4.1 | mg |
| Magnesium Stearate NF | 21.0 | mg |

Sodium lauryl sulfate is available from Cognis (Henkel). The colloidal silicon dioxide is either Cab-O—Sil®, available from Astro Chemicals Inc. or Aerosil 200®, available from Degussa. The pregelatinized starch is Starch 1500®, available from Colorcon. The sodium starch glycolate is Explotab®, available from Penwest Pharmaceuticals.

The azithromycin monohydrate, pregelatinized starch, microcrystalline cellulose, sodium lauryl sulfate, and colloidal silicon dioxide were mixed in a PMA high shear mixer for about 5 minutes to form a premix. Water was added to the premix and mixed in the PMA high shear mixer for about 10 minutes. Wet granules were discharged and placed on a tray which was placed in an oven at 55° C. for about 12 hours.

### EXAMPLE 2

### Preparation of Stabilized Azithromycin Monohydrate Tablets

The granules prepared in Example 1 were milled using a Quadro Co-mill equipped with a screen #75. Sodium starch glycolate was mixed with the granules using a tumble blender. Magnesium stearate was mixed with the granules using a tumble blender. The granules were compressed using a rotary high speed tablet press to form tablets which were coated with Opadry AMB.

### EXAMPLE 3

### Impurity Analysis of Azithromycin Monohydrate Granules

Wet granules prepared according to the procedure set forth in Example 1 were placed on a tray which was placed in an oven at 55° C. Six samples were taken at different times and the moisture content was determined using an OHAUS Balance. The water/moisture content of each sample varied from 3.3-12.5 wt %, based on the total weight of the granules. The samples were stored in glass bottles, sealed, and placed in an oven at 50° C. After 8 days, the samples were removed from the oven and the amount and type of impurities was determined by high performance liquid chromatography (HPLC).

Sample solutions were freshly prepared from azithromycin monohydrate and injected on column. The percentages of impurities was calculated from the integrator output. The performance of the HPLC system was tested using standardized solutions of azithromycin monohydrate.

Three impurities were identified and measured as a percentage of the total azithromycin monohydrate in each sample. Impurity I had a relative retention time of 0.47. Impurity II had a relative retention time of 0.55 (—N-demethyl-N-oxide). Impurity III had a retention time of 0.86 (N-demethyl). The results are summarized in Table 1.

TABLE 1

| Water | Impurity | Impurity | Impurity |
|---|---|---|---|
| Content | RRT 0.47 | RRT 0.55 | RRT 0.86 |
| 12.4% | 0.533 | 0.0522 | 0.013 |
| 8.8% | 0.633 | 0.033 | 0.013 |
| 6.9% | 0.55 | 0.0355 | 0.014 |
| 5.5% | 0.609 | 0.059 | 0.015 |
| 3.9% | 1.74 | 1.197 | 0.893 |
| 3.3% | 1.81 | 2.079 | 0.948 |
| | 0.517 | 0.069 | 0.055 |

The results in Table 1 show that azithromycin monohydrate has good chemical stability provided that the water content is maintained in the range of 5.5-12.4 wt %.

### EXAMPLE 4

### Preparation of a Azithromycin Monohydrate Granules

| Ingredient | Amount | |
|---|---|---|
| Azithromycin Monohydrate | 614.0 | mg |
| Pregelatinized Starch NF | 241.0 | mg |
| Microcrystalline Cellulose NF | 68.40 | mg |
| Sodium Lauryl Sulfate NF | 3.6 | mg |
| Colloidal Silicon Dioxide NF | 12.0 | mg |
| Purified Water | q.s. | |
| Sodium Starch Glycolate NF | 4.8 | mg |
| Magnesium Stearate NF | 25.20 | mg |

The azithromycin monohydrate, pregelatinized starch, microcrystalline cellulose, sodium lauryl sulfate, and colloidal silicon dioxide were mixed in a PMA high shear mixer for about 5 minutes to form a premix. Water was added to the premix and mixed in the PMA high shear mixer for about 10 minutes. Wet granules were discharged and placed on a tray which was placed in an oven at 55° C. for about 12 hours to achieve an LOD or water content of 6-7%.

The granules were milled using a Quadro Co-mill equipped with a screen #75. Sodium starch glycolate was mixed with the granules using a tumble blender. Magnesium stearate was mixed with the granules using a tumble blender.

### EXAMPLE 5

Determination of the relative humidity at which azithromycin monohydrate granules (600 mg) reach an equilibrium moisture content of 6-7%.

The granules prepared in Example 4 having a water content of 6-7% were placed in an automated moisture balance, DVS-1000, supplied by Surface Measurement Systems (London, UK). An incubator temperature of 25° C. was maintained throughout the experiment. The moisture sorption-desorption isotherm was generated using approximately 50 mg of granules that were weighed into a round bottomed quartz pan. The humidity program incremented in 10% RH steps starting at 0% RH and ending at 90% RH and back to 0% RH. An equilibrium criteria of 0.001 wt % per 5-minute interval was used.

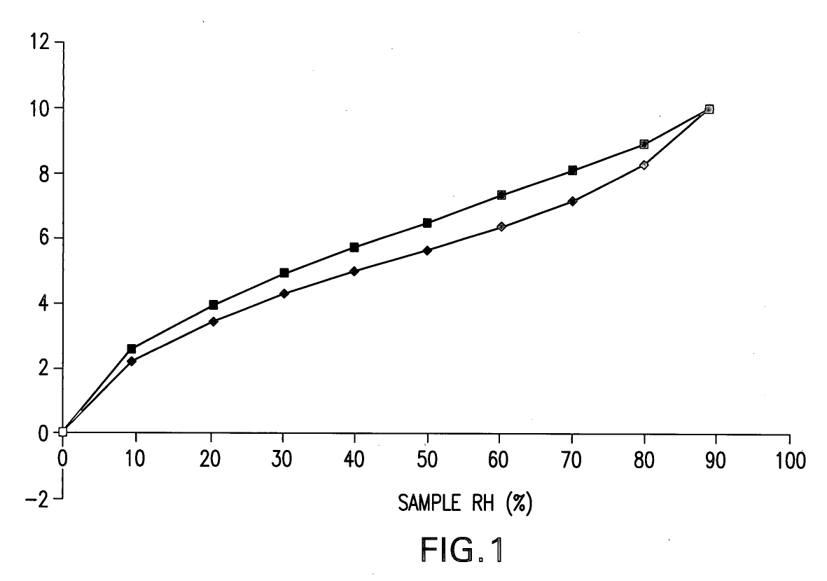

With reference to the drawings, FIG. 1 is a graph illustrating moisture sorption-desorption isotherm of the azithromycin monohydrate granules FIG. 1 shows that the granules picks up and lose water without significant hysteresis. In addition, the relative humidity at which the granules equilibrate to between 6-7% moisture is approximately 60% RH.

### EXAMPLE 6

Evaluation of the effect of humidity on azithromycin monohydrate granules (600 mg).

Approximately 50 mg of the azithromycin monohydrate granules prepared in Example 4 were equilibrated to 60% RH and upon equilibration, the humidity was decreased to 10%. The granules remained at this humidity until an equilibrium criteria of 0.001% was met. Then the sample humidity was raised to 60% RH. This schedule was repeated from 30% RH to 70% RH in 5% RH increments. Between each relative humidity, a 60% RH equilibration step was inserted to return the sample back to a target loss on drying or water content of approximately 6.3%.

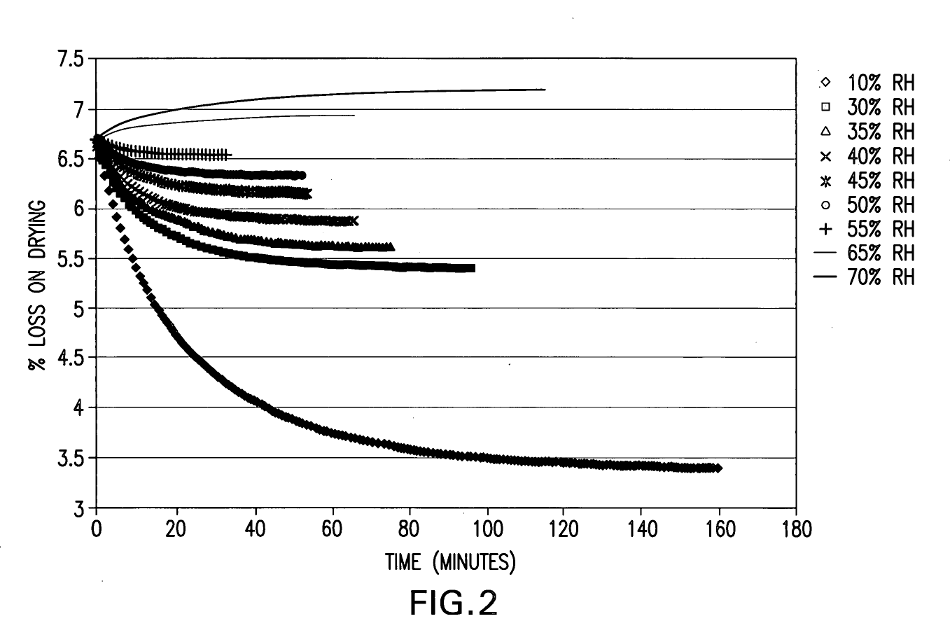

With reference to the drawings, FIG. 2 is a graph illustrating the percent LOD vs. time for the azithromycin monohydrate granules according to the schedule set form in this example. FIG. 2 shows that the granules equilibrated to their desired equilibrium moisture content in approximately 30 minutes, and that the lower the humidity the longer time the granules required to equilibrate to that humidity. The equilibrium moisture content of the granules is summarized in Table 2.

TABLE 2
Equilibrium Moisture Content of Azithromycin
Monohydrate Granules (600 mg)

| Relative Humidity (25° C.) | Equilibrium Moisture Content |
|---|---|
| 10% | 3.40 |
| 30% | 5.40 |
| 35% | 5.63 |
| 40% | 5.88 |
| 45% | 6.15 |
| 50% | 6.33 |
| 55% | 6.53 |
| 60% | 6.63 |
| 65% | 6.93 |
| 70% | 7.19 |

To further illustrate the dramatic effect of moisture loss, the first 10 minutes of moisture loss data were plotted against time. The slope of each line was calculated using regression analysis (R² ranged from 0.987-0.961) to determine the rate of moisture loss (or uptake). The rates are summarized in Table 3 which shows that the granules lose water rapidly at 10% RH. The rate was determined to be 0.127% per minute.

TABLE 3
Rate of Moisture Loss (Uptake) when azithromycin monohydrate
granules (600 mg) are dried to a target LOD of 6.63% and
exposed to the indicated humidity at 25° C.

| Relative Humidity (25° C.) | Rate of Moisture Loss (Uptake) |
|---|---|
| 10% | 0.127% per minute |
| 30% | 0.066% per minute |
| 35% | 0.059% per minute |
| 40% | 0.048% per minute |
| 45% | 0.034% per minute |
| 50% | 0.023% per minute |
| 55% | 0.011% per minute |
| 60% | Maintains equilibrium moisture content |
| 65% | (0.012% per minute) |
| 70% | (0.021% per minute) |

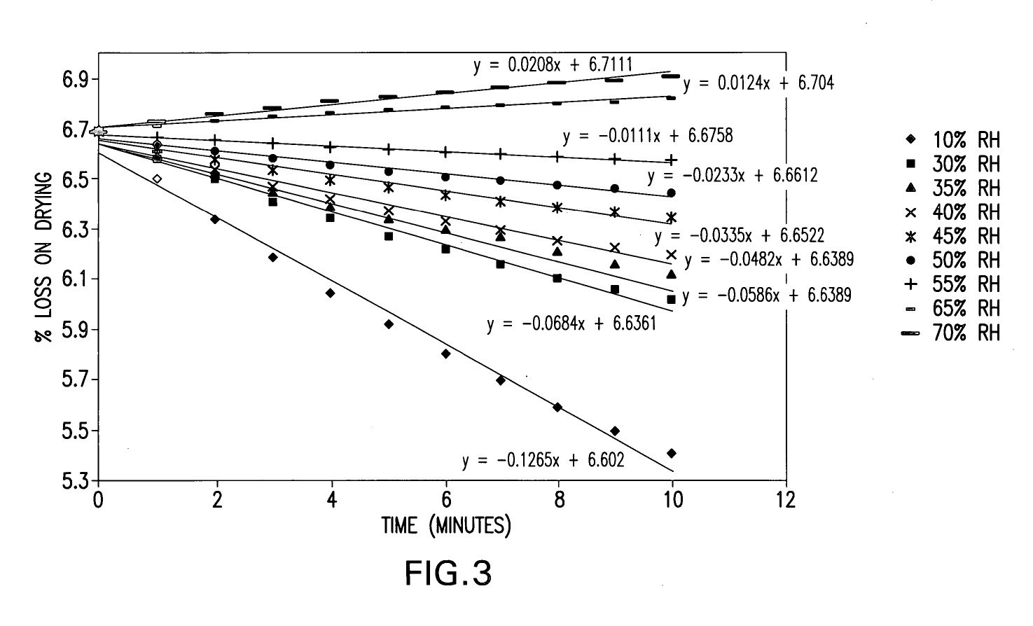

With reference to the drawings, FIG. 3 is a graph illustrating the percent LOD vs. time for azithromycin monohydrate granules upon exposure to different humidity levels.

Thus, the results in Tables 2 and 3, and FIGS. 2 and 3 show that in order to maintain a water content or LOD of between 6% and 7% in azithromycin monohydrate granules, a humidity range of between 40% RH and 70% RH should be maintained during manufacturing operations.

While the invention has been described with particular reference to certain embodiments thereof, it will be understood that changes and modifications may be made by those of ordinary skill within the scope and spirit of the following claims:

1: A stabilized azithromycin composition comprising azithromycin monohydrate, and 7 to about 15 weight percent, based on the total weight of the composition, of water.

2: A The stabilized azithromycin composition of claim 1 comprising azithromycin monohydrate, and 7 to about 12.4 weight percent, based on the total weight of the composition, of water.

3: The composition according to claim 2, wherein the water is present in an amount of from 7 to about 8 weight percent, based on the total weight of the composition.

4: The composition according to claim 3, wherein the water is present in an amount of 7 weight percent, based on the total weight of the composition.

5: The composition according to claim 1, which is in the form of an oral dosage form selected from the group consisting of tablets, granules, dragees, hard or soft capsules, powders, multiparticules, and combinations thereof.

6: The composition according to claim 5, wherein the oral dosage form is a tablet.

7: The composition according to claim 1, wherein the azithromycin monohydrate is present in an amount of from about 0.1 to about 95 weight percent, based on the total weight of the composition.

8: The composition according to claim 7, wherein the azithromycin monohydrate is present in an amount of from about 30 to about 85 weight percent.

9: The composition according to claim 8, wherein the azithromycin monohydrate is present in an amount of from about 50 to about 75 weight percent.

10: The composition according to claim 1, which is essentially free of an antioxidant.

11: A dosage form according to claim 1, comprising about 60 weight percent of azithromycin monohydrate; and 7 weight percent of water, wherein the weight percents are based on the total weight of the composition.

12. (canceled)

13: A method for preparing a stabilized azithromycin composition comprising mixing azithromycin monohydrate and at least one excipient containing water to form a stabilized azithromycin composition having a water content from 7 to about 15 weight percent, based on the total weight of the composition.

14: The method according to claim 12, wherein the method is conducted within a humidity range of 20-99% relative humidity.

15: The method according to claim 14, wherein the relative humidity is 25-90%.

16: The method according to claim 15, wherein the relative humidity is 30-80%.

17: The method according to claim 16, wherein the relative humidity is 45-70%.

18: A method for preparing a stabilized azithromycin composition comprising: (a) mixing azithromycin monohydrate, and optionally one or more excipients, to form a premix; (b) adding water, and optionally one or more excipients, to the premix formed in Step (a) to form a mixture; (c) drying the mixture formed in Step (b), and optionally milling the mixture; and (d) adding water to the mixture formed in Step (c) to form a stabilized azithromycin composition having a water content from 7 to about 15 weight percent, based on the total weight of the composition.

19: A method for treating a bacterial infection comprising administering to a mammal in need of such treatment, a therapeutically effective amount of a stabilized azithromycin composition according to claim 1.

20: A therapeutic package comprising a container and a stabilized azithromycin composition according to claim 1.
